(19) United States
(12) Patent Application Publication
(10) Pub. No.: US 2025/0059140 A1
(43) Pub. Date: Feb. 20, 2025

(54) C19 DITERPENOID ALKALOID, AND PREPARATION METHOD AND APPLICATION THEREOF

(71) Applicant: SOUTHWEST JIAOTONG UNIVERSITY, Chengdu (CN)

(72) Inventors: Xianli ZHOU, Chengdu (CN); Shuai HUANG, Chengdu (CN); Lin CHEN, Chengdu (CN); Feng GAO, Chengdu (CN)

(21) Appl. No.: 18/814,472

(22) Filed: Aug. 24, 2024

Related U.S. Application Data
(63) Continuation of application No. PCT/CN2023/072275, filed on Jan. 16, 2023.

(30) Foreign Application Priority Data
Feb. 25, 2022 (CN) 202210181794.0

(51) Int. Cl. C07D 221/24; A61K 36/714; A61P 9/04

(52) U.S. Cl. CPC C07D 221/24; A61K 36/714; A61P 9/04; A61K 2236/333

(57) ABSTRACT

A C₁₉ diterpenoid alkaloid represented by

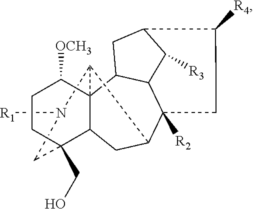

where R₁ is hydrogen, methyl or ethyl; R₂ is hydroxyl, methoxy or ethoxy; R₃ is hydroxyl or methoxy; and R₄ is hydroxyl or methoxy. The C₁₉ diterpenoid alkaloid is prepared from a plant material belonging to the genus Aconitum of the family Ranunculaceae through extraction, concentration and separation using silica gel column chromatography. An application of the C₁₉ diterpenoid alkaloid in the preparation of cardiotonic drugs and drugs for preventing and/or treating heart failure is also provided.

### CROSS-REFERENCE TO RELATED APPLICATIONS

This application is a continuation of International Patent Application No. PCT/CN2023/072275, filed on Jan. 16, 2023, which claims the benefit of priority from Chinese Patent Application No. 202210181794.0, filed on Feb. 25, 2022. The content of the aforementioned application, including any intervening amendments thereto, is incorporated herein by reference in its entirety.

### TECHNICAL FIELD

This application relates to pharmaceuticals, and more specifically to a C₁₉ diterpenoid alkaloid, and a preparation method and application thereof.

### BACKGROUND

Heart failure is a serious clinical syndrome derived from various cardiovascular diseases. Traditionally, it is believed that the heart failure is mainly caused by the abnormal myocardial contractility, which will make the cardiac output fail to meet the needs of the body, thereby resulting in a series of symptoms and signs. Cardiotonic drugs are important for the treatment and prevention of heart failure, which mainly include digitalis drugs and non-digitalis drugs. For the past 200 years, the most commonly used in the treatment of heart failure has been digitalis. The digitalis selectively inhibits the activity of cardiomyocyte membrane Na⁺-K⁺-ATPase and increases the internal flow of Ca²⁺ through a biphasic Na⁺—Ca²⁺ exchange mechanism, thereby increasing the Ca²⁺ concentration in the cytoplasm and exerting a positive inotropic effect. However, the digitalis may cause a variety of adverse effects, including cardiac arrhythmias (e.g., ventricular premature contractions and conduction block), gastrointestinal symptoms (e.g., nausea and vomiting), and neurologic deficits (e.g., dizziness, yellow vision, and green vision). The non-digitalis drugs mainly include dopamine, dobutamine, amrinone, milrinone, and levosimendan. The non-digitalis drugs can increase myocardial contractility while accelerating the heart rate of patients and increasing myocardial oxygen consumption, thus aggravating myocardial ischemia.

With the aggravation of myocardial ischemia, myocardial contractility will also be reduced, which will form a dependence on non-digitalis drugs, leading to a vicious circle.

Aconitum plants, belonging to the family Ranunculaceae, have a long history of medicinal use, which are rich in resources, and have about 350 species in the world. It is distributed in the temperate zone in the northern hemisphere, mainly in Asia, followed by Europe and North America. In China, there are about 200 species of Aconitum plants, 76 of which are available for medicinal use, and are mainly distributed in the southwestern Hengduan Mountainous area, such as alpine zones of northern Yunnan, western Sichuan, and eastern Tibet. The medicinal value of Aconitum plants has been widely documented. For example, as recorded in the Chinese Pharmacopoeia (2020), Aconitum carmichaeli and Aconitum kusnezoffii have the efficacy of dispelling wind and dampness and warming the channel to alleviate pain, and Aconiti Lateralis Radix Praeparata has the efficacy of restoring yang to rescue collapse, replenishing fire to help yang, and dispersing cold to alleviate pain. The traditional Chinese medicine compound formulas based on the Aconiti Lateralis Radix Praeparata, e.g., Fuzilizhong pills and Sini decoction, are also included in the Chinese Pharmacopoeia, both of which have the effects of warming the middle and strengthening the spleen, dispelling cold, and restoring yang to rescue collapse.

As a characteristic component of plants from the genus Aconitum, diterpenoid alkaloid has been found to possess a variety of biological activities, such as anti-inflammatory, anti-arrhythmic, cardiotonic, analgesic, antitumor, and inhibition of acetylcholinesterase. In recent years, several C₁₉-diterpenoid alkaloids with cardiotonic and anti-heart failure activities (such as mesaconine) have been successfully separated from Aconiti Lateralis Radix Praeparata by activity tracking (Wang Fengpeng et al.). However, the cardiotonic effect of mesaconine is still insufficicent, and thus it is of great importance to develop a new compound with more excellent cardiotonic effect for the clinical prevention and treatment of heart failure.

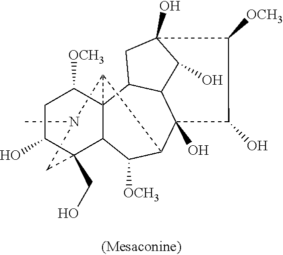

### SUMMARY

A first objective of the present disclosure is to provide a C₁₉ diterpenoid alkaloid and its preparation method.

A second objective of the present disclosure is to provide applications of the C₁₉ diterpenoid alkaloid in the preparation of a cardiotonic drug, and drugs for preventing and/or treating heart failure.

In a first aspect, this application provides a compound of formula (I), or a pharmaceutically-acceptable salt thereof:

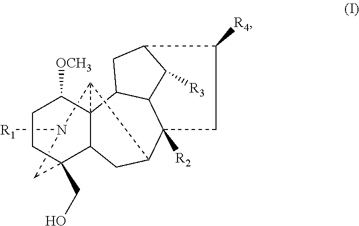

wherein R₁ is hydrogen, methyl or ethyl; R₂ is hydroxyl, methoxy or ethoxy; R₃ is hydroxyl or methoxy; and R₄ is hydroxyl or methoxy.

In some embodiments, the compound is selected from the group consisting of:

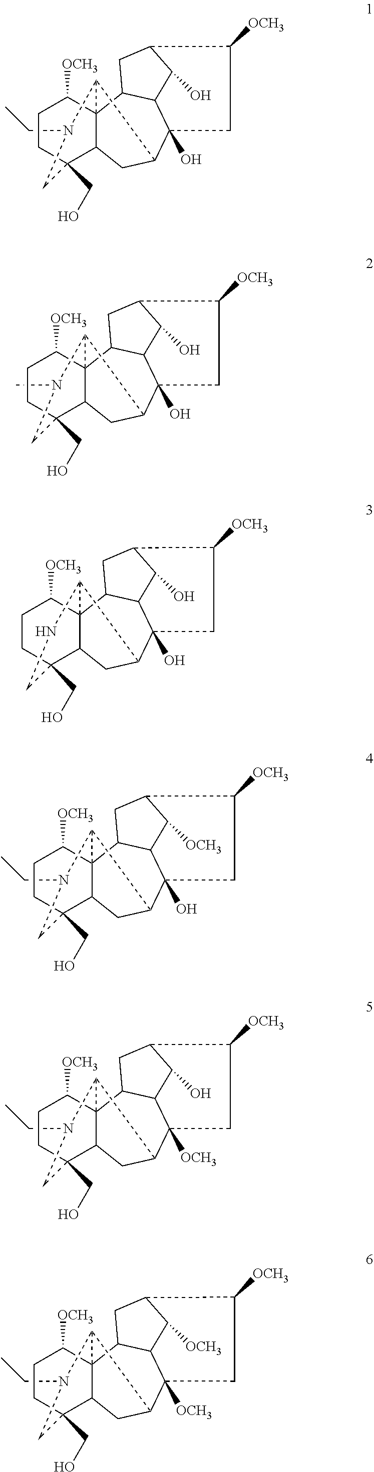

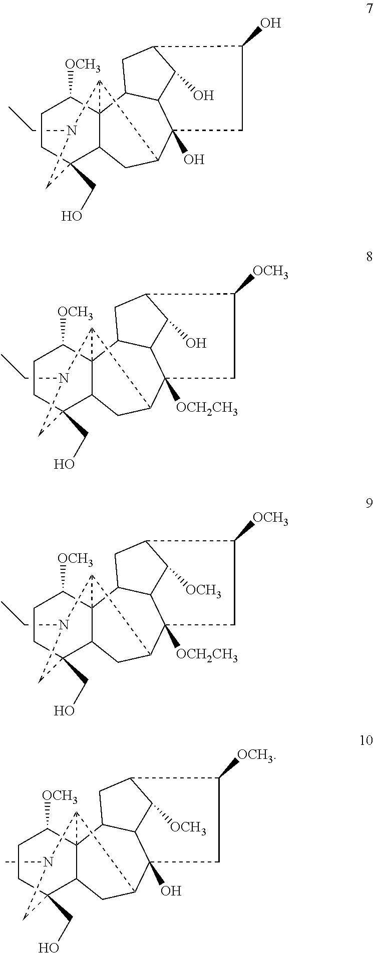

In a second aspect, this application provides a method of preparing the above-mentioned compound, comprising:
(a) subjecting a plant material belonging to genus Aconitum of family Ranunculaceae to extraction with ethanol, and concentration to obtain an extract;
(b) dissolving the extract in water, followed by adjustment to pH 1-3 and extraction sequentially with petroleum ether and ethyl acetate to obtain an aqueous phase;
(c) adjusting the aqueous phase to pH 9.5-11.0, followed by extraction with dichloromethane and concentration to obtain a first alkaloid sample;
(d) refluxing the first alkaloid sample with a solution of 5 wt. % NaOH in methanol for 2 h, followed by adjustment to pH 8-9 and concentration under reduced pressure to obtain a second alkaloid sample;
(e) subjecting the second alkaloids sample to normal-phase silica gel column chromatography in a gradient elution, and combining eluate fractions in sequence to respectively obtain a first crude product, a second crude product and a third crude product, wherein the eluate fractions are detected through thin-layer chromatography, and an eluent used in the normal-phase silica gel column chromatography is a mixed solution of CH₂Cl₂ and CH₃OH with a volume ratio of 1-0:0-1; and
(f) separately subjecting the first crude product, the second crude product and the third crude product to normal phase silica gel column chromatography for separation and purification to obtain the compound.

In an embodiment, in step (a), a concentration of the ethanol is 95 wt. %.

In an embodiment, in step (a), the plant material is Aconitum apetalum (Huth) B. Fedtsch.; and
in step (f), a mobile phase used in the normal phase silica gel column chromatography of the first crude product is a mixture of CH₂Cl₂, MeOH and diethylamine in a volume ratio of 100-10:1:0.1, and the obtained compound is compound 8 represented by

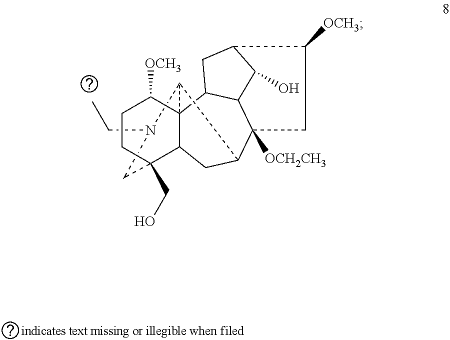

a mobile phase used in the normal phase silica gel column chromatography of the second crude product is a mixture of CH₂Cl₂, MeOH and diethylamine in a volume ratio of 100-10:1:0.1, and the obtained compound is a combination of compound 1, compound 2 and compound 3, respectively represented by

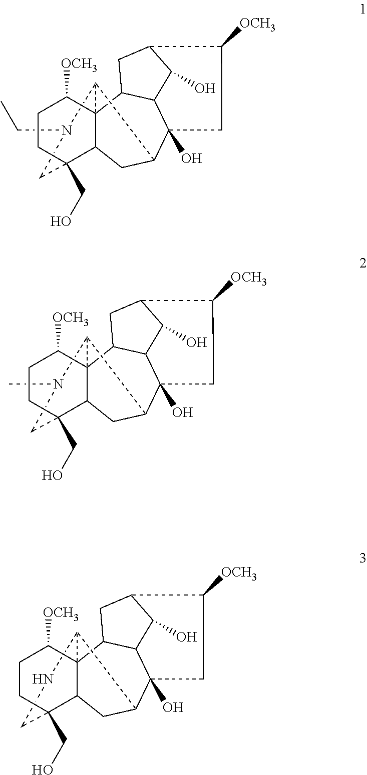

and
a mobile phase used in the normal phase silica gel column chromatography of the third crude product is a mixture of CH₂Cl₂, MeOH and diethylamine in a volume ratio of 100-10:1:0.1, and the obtained compound is compound 7 represented by

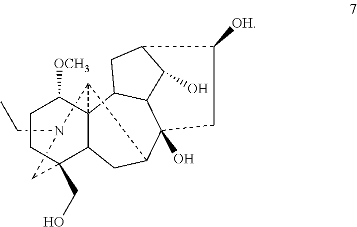

In an embodiment, in step (a), the plant material is Aconitum brevicalcaratum (Finet & Gagnep.) Diels;
in step (f), a mobile phase used in the normal phase silica gel column chromatography of the first crude product is a mixture of CH₂Cl₂, MeOH and diethylamine in a volume ratio of 100-10:1:0.1, and the obtained compound is a combination of compound 4, compound 5 and compound 6, respectively represented by

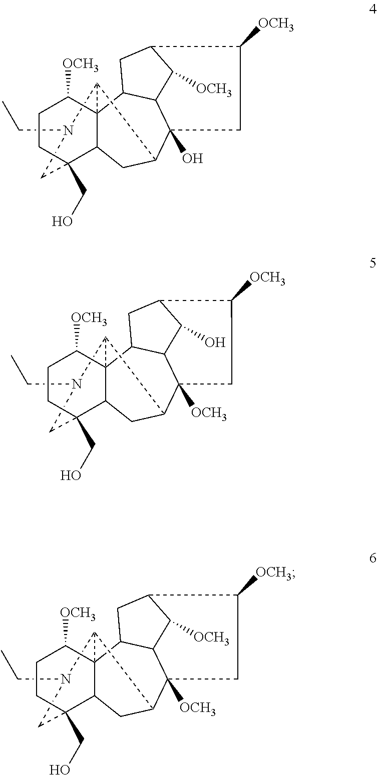

and
a mobile phase used in the normal phase silica gel column chromatography of the second crude product is a mixture of CH₂Cl₂, MeOH and diethylamine in a volume ratio of 100-10:1:0.1, and the obtained compound is a combination of compound 9 and compound 10, respectively represented by

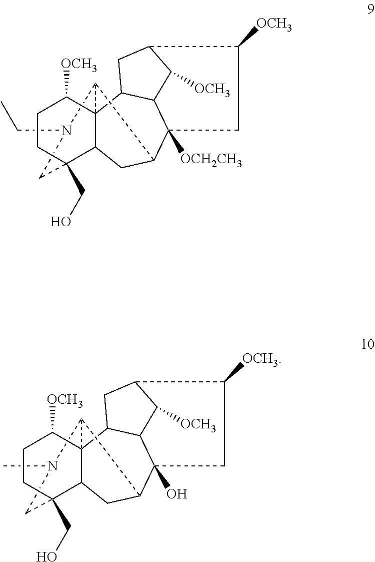

In a third aspect, this application provides a pharmaceutical composition, comprising:
the afore-mentioned compound or a pharmaceutically acceptable salt thereof as an active ingredient; and
a pharmaceutically-acceptable excipient or adjuvant.

In an embodiment, the pharmaceutical composition is in a form of oral preparation.

In a fourth aspect, this application provides a method for strengthening heart in a subject in need thereof, comprising:
administering a therapeutically effective amount of the aforementioned compound or a pharmaceutically-acceptable salt thereof to the subject.

In a fifth aspect, this application provides a method for preventing and and/or treating heart failure in a subject in need thereof, comprising:
administering a therapeutically effective amount of the aforementioned compound or a pharmaceutically-acceptable salt thereof to the subject.

Definitions of terms relating to the present disclosure are described below.

The term “pharmaceutically acceptable” refers to that a carrier, transporter, diluent, excipient, and/or a formed salt is generally chemically or physically compatible with the other ingredients to form a pharmaceutical dosage and is physiologically compatible with the receptor.

The term “pharmaceutically acceptable salt” refers to acid and/or base salts formed by the afore-mentioned compound or stereoisomers thereof with inorganic and/or organic acids and bases, and also includes amphoteric salts (inner salts) and quaternary ammonium salts, such as alkylammonium salts. These salts may be directly obtained in the final isolation and purification process of the compounds, or may also be obtained by mixing the afore-mentioned compound or stereoisomers thereof with a suitable (e.g. equivalent) quantity of acid or base. These salts may be collected in a form of precipitate through filtration, or obtained by recovery through evaporation of the solvent, or prepared by freeze-drying after reaction in an aqueous medium. The salts described in the present disclosure may be hydrochloride, sulfate, citrate, benzenesulfonate, hydrobromide, hydrofluoric acid, phosphate, acetate, propionate, succinate, oxalate, malate, succinate, fumarate, maleate, tartrate, or trifluoroacetate of the compound.

The modes of administration of the compounds or pharmaceutical compositions of the present disclosure are not particularly limited, and representative modes of administration include (but are not limited to): oral administration, parenteral (intravenous, intramuscular, or subcutaneous) administration, and topical administration.

Solid dosage forms for oral administration include capsules, tablets, pills, powder, and granules. In these solid dosage forms, the active compound is mixed with at least one conventional inert excipient (or carrier), such as sodium citrate or dicalcium phosphate, or with the following ingredients: (a) a fillers or bulking agent, such as, starch, lactose, sucrose, dextrose, mannitol, and silicic acid; (b) a binder, such as hydroxymethyl cellulose, alginate, gelatin, polyvinylpyrrolidone, sucrose, and Arabic gum; (c) a humectant, such as glycerin; (d) a disintegrant, e.g., agar, calcium carbonate, potato starch or tapioca starch, alginate, a complex silicate, and sodium carbonate; (e) a retardant, e.g., paraffin wax; (f) an absorption accelerator, e.g., quaternary amine compounds; (g) a wetting agent, e.g., cetearyl alcohol and glycerol monostearate; (h) an adsorbent, e.g., kaolin; and (i) a lubricant, e.g., talc, calcium stearate, magnesium stearate, solid polyethylene glycol, sodium dodecyl sulfate, or mixtures thereof. In capsules, tablets and pills, the dosage form may also contain a buffering agent.

Solid dosage forms such as tablets, sugar pills, capsules, pills and granules may be prepared with coatings and shell materials such as sausage casings and other materials well known in the art. They may include an opacifying agent, and the active compound or compounds in such compositions may be released in a delayed manner in a portion of the digestive tract. Polymeric substances and wax-like substances may be used for component encapsulation. If necessary, the active compound may also be formed in the form of a microcapsule with one or more of the excipients mentioned above.

Liquid dosage forms for oral administration include pharmaceutically acceptable emulsions, solutions, suspensions, syrups or tinctures. In addition to the active compound, the liquid dosage forms may include inert diluents commonly used in the art, such as water or other solvents, solubilizers and emulsifiers, e.g., ethanol, isopropanol, ethyl carbonate, ethyl acetate, propylene glycol, 1,3-butylene glycol, dimethylformamide, and oils, particularly, cottonseed oil, peanut oil, corn embryo oil, olive oil, castor oil, and sesame oil or mixtures thereof.

In addition to the inert diluents, the compositions may also include auxiliaries such as wetting agents, emulsifiers and suspending agents, sweeteners, corrigents and fragrances.

In addition to the active compounds, the suspensions may include suspending agents such as, ethoxylated isooctadecanol, polyoxyethylene sorbitol and dehydrated sorbitan esters, microcrystalline cellulose, methanolic aluminum, agar or mixtures thereof.

Compositions for parenteral administration may include physiologically acceptable sterile aqueous or non-aqueous solutions, dispersions, suspensions or emulsions, and sterile powders for re-dissolution into sterile injectable solutions or dispersions. Suitable aqueous and non-aqueous carriers, diluents, solvents or excipients include water, ethanol, polyols and suitable mixtures thereof.

Pharmaceutically acceptable excipients as described herein refer to substances other than the active ingredient included in the dosage form.

The pharmaceutically acceptable auxiliary ingredients described in the present disclosure have a certain physiological activity. However, these ingredients will not change the dominant position of the pharmaceutical composition during the disease treatment, but only exert auxiliary effects, which are the known effects of these ingredients and are common auxiliary therapeutic modes in the art. If the aforementioned auxiliary compositions are used in conjunction with the pharmaceutical composition of the present disclosure, it shall still fall within the scope of protection of the present disclosure.

Pharmacodynamic experiments have shown that the compounds of the present disclosure all have cardiotonic effects. In particular, compared with the positive control group (mesaconine), the cardiotonic effect of compounds 1, 2, 4, 7 and 10 of the present disclosure is significantly enhanced. The compounds of the present disclosure can be used to prepare cardiotonic drugs, and drugs for preventing and/or treating heart failure.

The compounds of the present disclosure can be obtained from Aconitum plants through extraction and isolation, which are wide in source and have broad clinical application prospects and market prospects.

Obviously, according to the above descriptions of the present disclosure, in accordance with the ordinary technical knowledge and common means in the art, various forms of modifications, substitutions or changes can be made without departing from the above basic technical ideas of the present disclosure.

The present disclosure will be further explained with reference to the following specific embodiments. However, it should not be understood that the scope of the present disclosure is limited to the following examples. Any implement realized based on the above contents of the present disclosure shall be included in the scope of the present disclosure.

### BRIEF DESCRIPTION OF THE DRAWINGS

FIG. 1A shows H9c2 cell viability results tested by MTT assay, where the H9c2 cells are treated respetively with different concentrations of compound 1 for 4 h, 50 μM Trimetazidine for 4 h and 800 μM CoCl₂ for 20 h;

FIGS. 1B-C show an effect of the compound 1 on the Na⁺/K⁺-ATPase specific activity and Ca²⁺/Mg²⁺-ATPase activity of the hypoxic cardiomyocytes, where the test is performed in triplicate; ##P<0.01, ###P<0.001, and ####P<0.0001, indicating the significant difference from the control group; and *P<0.05, **P<0.01, ***P<0.001, and ****P<0.0001, indicating the significant difference from the CoCl₂ group;

FIGS. 1D-I show fluorescence detection results of reactive oxygen species (ROS) of different groups of H9c2 cells (scale bar: 20 μm), where 1D: control group; 1E: CoCl₂ group; 1F: 50 μM-Trimetazidine group; 1G: 12.5 μM-Cammaconine group; 1H: 25 μM-Cammaconine group; and 11:50 μM-Cammaconine group;

FIG. 2A shows transcriptional level results (detected by q-PCR) of a key hypoxia indicator HIF-1α in cardiomyocytes; and

FIGS. 2B-F show transcriptional level results (detected by q-PCR) of calcium channel-related indicators in cardiomyocytes, where 2B: DHPR; 2C: RyR2; 2D: SERCA2a; 2E: NCX; and 2F: NHE.

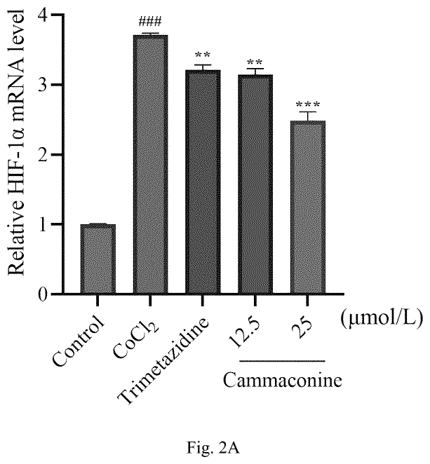

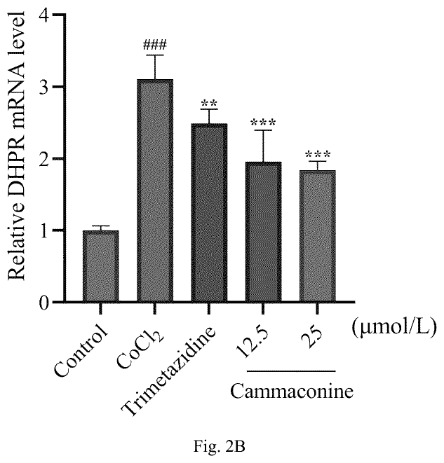

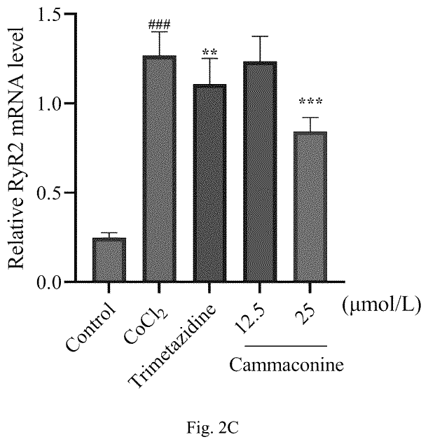

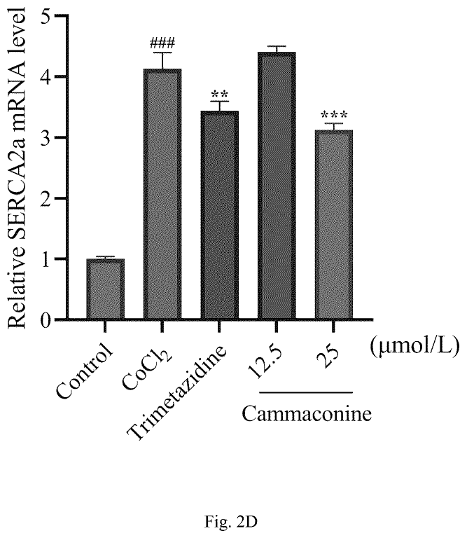

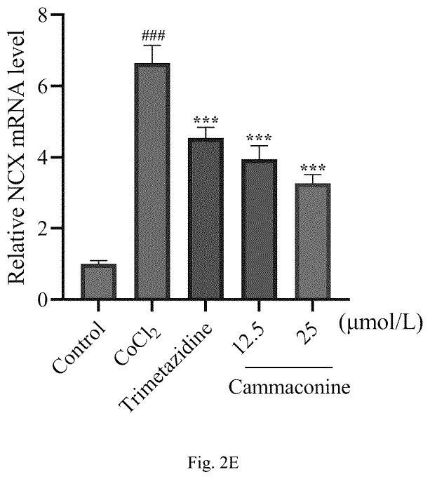

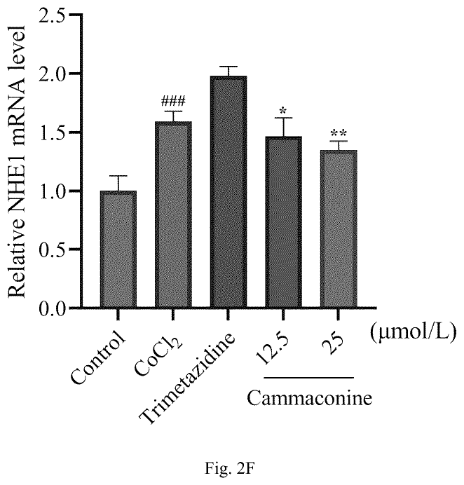

In FIGS. 2A-F, the detection is performed in triplicate; ##P<0.01, ###P<0.001, and ####P<0.0001, indicating the significant difference from the control group; and *P<0.05, **P<0.01, ***P<0.001, and ****P<0.0001, indicating the significant difference from the CoCl₂ group.

### DETAILED DESCRIPTION OF EMBODIMENTS

The raw materials and instruments used in embodiments of the present disclosure are commercially available.

### Embodiment 1 Preparation of Compounds 1-10

### 1. Preparation of Compounds 1, 2, 3, 7 and 8

10 kg of a dried root of Aconitum apetalum (Huth) B. Fedtsch. was subjected to extraction in 95 wt. % ethanol at room temperature for three times, each lasting for 7 days. The extracted solutions were combined and concentrated under reduced pressure to obtain an extract (510 g).

The extract was dissolved in 50° C. water, adjusted to pH 3.0 with a 10% (w/w) hydrochloric acid solution and subjected to extraction with petroleum ether and ethyl acetate sequentially to obtain an aqueous phase. The aqueous phase was adjusted to pH 9.4 with a 24% (w/w) concentrated ammonia water, and subjected to extraction with dichloromethane and concentration under reduced pressure to obtain a first alkaloid sample (90 g).

The first alkaloid sample was refluxed with a solution of 5 wt. % NaOH in methanol for 2 h, adjusted to pH 8-9 and concentrated under reduced pressure to recover methanol and obtain a second alkaloid sample.

The second alkaloid sample was subjected to normal-phase silica gel column chromatography in a gradient elution mode, and eluate fractions were combined in sequence to obtain a first crude product, a second crude product and a third crude product, where the eluate was monitored by thin-layer chromatography, and an eluent used in the normal-phase silica gel column chromatography was a mixed solution of CH₂Cl₂ and CH₃OH with a volume ratio of 1-0:0-1.

The first crude product was subjected to normal phase silica gel column chromatography using a mobile phase of a mixture of CH₂Cl₂, MeOH and diethylamine in a volume ratio of 100-10:1:0.1 for separation and purification to obtain compound 8 (10 mg), which was struturally identified and characterized by nuclear magnetic resonance (NMR).

The second crude product was subjected to normal phase silica gel column chromatography using a mobile phase of a mixture of CH₂Cl₂, MeOH and diethylamine in a volume ratio of 100-10:1:0.1 for separation and purification to obtain compound 1 (13 mg), compound 2 (17 mg) and compound 3 (11 mg), which were structurally identified and characterized by NMR.

The third crude product was subjected to normal phase silica gel column chromatography using a mobile phase of a mixture of CH₂Cl₂, MeOH and diethylamine in a volume ratio of 100-10:1:0.1 for separation and purification to obtain compound 7 (22 mg), which were structurally identified and characterized by NMR.

### 2. Preparation of Compounds 4, 5. 6, 9 and 10

5.0 kg of a dried root of Aconitum brevicalcaratum (Finet & Gagnep.) Diels was subjected to soaking extraction in 95 wt. % ethanol at room temperature for three times, each lasting for 7 days. The extracted solutions were combined and concentrated under reduced pressure to obtain an extract (310 g).

The extract was dissolved in 50° C. water, adjusted to pH 3.0 with a 10% w/w hydrochloric acid solution and subjected to extraction with petroleum ether and ethyl acetate sequentially to obtain an aqueous phase. The aqueous phase was adjusted to pH 9.4 with a 24% w/w concentrated ammonia water, and subjected to extraction with dichloromethane and concentration under reduced pressure to obtain a first alkaloid sample (30 g).

The first alkaloid sample was refluxed with a solution of 5 wt. % NaOH in methanol for 2 h, adjusted to pH 8-9 and concentrated under reduced pressure to recover the methanol solution and obtain a second alkaloid sample.

The second alkaloid sample was subjected to normal-phase silica gel column chromatography in a gradient elution mode, and eluate fractions were combined in sequence to obtain a first crude product, a second crude product and a third crude product, where the eluate was monitored by thin-layer chromatography, and an eluent used in the normal-phase silica gel column chromatography was a mixed solution of CH₂Cl₂ and CH₃OH with a volume ratio of 1-0:0-1.

The first crude product was subjected to normal phase silica gel column chromatography using a mobile phase of a mixture of CH₂Cl₂, MeOH and diethylamine in a volume ratio of 100-10:1:0.1 for separation and purification to obtain compound 4 (5 mg), compound 5 (8 mg) and compound 6 (6 mg), which was struturally identified and characterized by NMR.

The second crude product was subjected to normal phase silica gel column chromatography using a mobile phase of a mixture of CH₂Cl₂, MeOH and diethylamine in a volume ratio of 100-10:1:0.1 for separation and purification to obtain compound 9 (15 mg) and compound 10 (18 mg), which were structurally identified and characterized by NMR.

Compounds 1-10 prepared in the present disclosure were all C₁₉ diterpene alkaloids, and their chemical structures and physico-chemical constants were listed below.

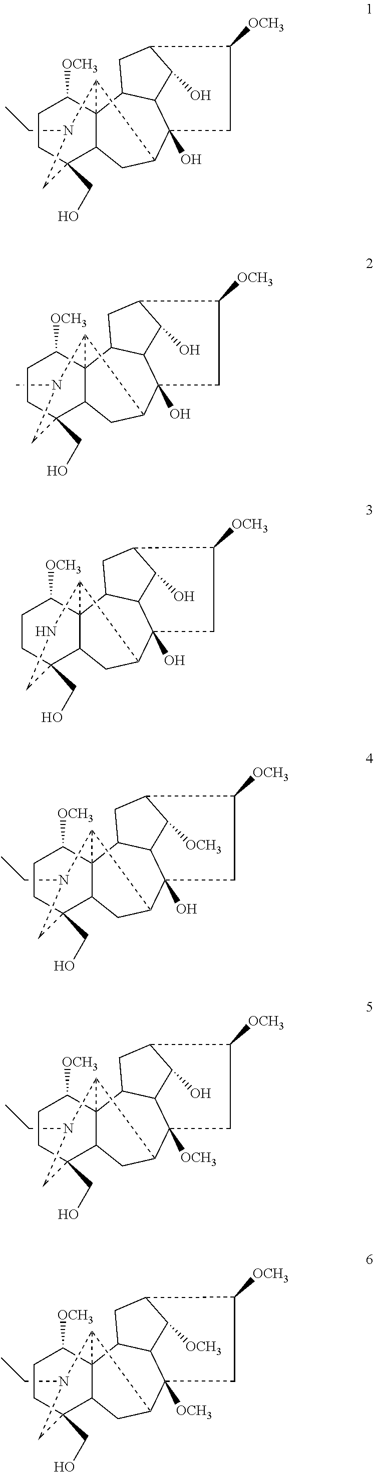

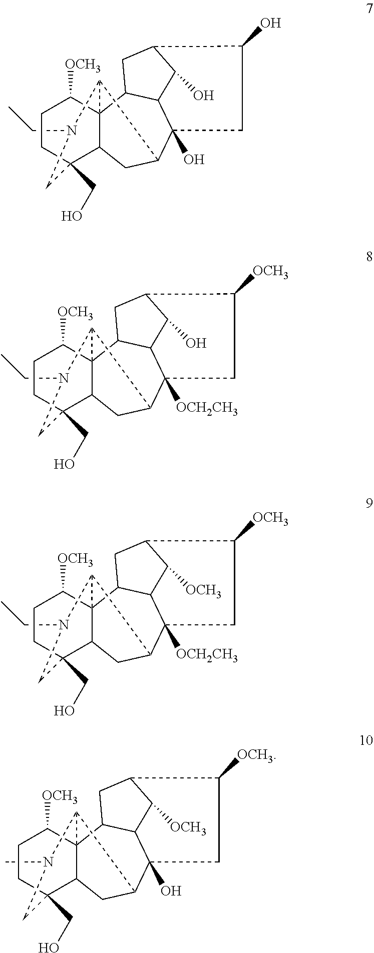

Compound 1: cammaconine, which was white amorphous powder and reacted positively with bismuth potassium iodide.

¹H-NMR (CDCl₃, 400 MHz) δ: 1.06 (3H, t, J=7.2 Hz, NCH2CH3), 3.34, 3.27 (each 3H, s, 2×OCH₃), 3.04 (1H, dd, J=10.8 Hz, 6.6 Hz, H-10), 4.14 (1H, t, J=4.8 Hz, H-14β), 3.16 (1H, m, H-16α).

¹³C-NMR (CDCl₃, 100 MHz) δ: 86.4 (d, C-1), 25.8 (t, C-2), 32.2 (t, C-3), 39.2 (s, C-4), 46.0 (d, C-5), 24.7 (t, C-6), 45.9 (d, C-7), 73.0 (s, C-8), 47.0 (d, C-9), 45.6 (d, C-10), 48.9 (s, C-11), 27.8 (t, C-12), 37.6 (d, C-13), 75.7 (d, C-14), 38.4 (t, C-15), 82.3 (d, C-16), 63.2 (d, C-17), 69.0 (t, C-18), 53.1 (t, C-19), 49.6 (t, C-21), 13.8 (q, C-22), 56.7 (q, 1-OCH₃), 56.5 (q, 16-OCH₃).

Compound 2: N-deethyl-N-methyl cammaconine, which was white amorphous powder and reacted positively with bismuth potassium iodide.

¹H-NMR (CDCl₃, 400 MHz) δ: 3.10 (1H, m, H-10), 4.14 (1H, t, J=4.8 Hz, H-14β), 3.43 (1H, m, H-16a), 3.25, 3.38 (each 1H, ABq, J=11.2 Hz, H-18), 3.31, 3.37 (each 3H, s, 2×OCH₃), 2.30 (3H, s, NCH₃).

¹³C-NMR (CDCl₃, 100 MHz) δ:86.4 (d, C-1), 26.0 (t, C-2), 32.2 (t, C-3), 39.3 (s, C-4), 45.1 (d, C-5), 24.7 (t, C-6), 45.0 (d, C-7), 73.0 (s, C-8), 47.1 (d, C-9), 45.6 (d, C-10), 48.9 (s, C-11), 27.7 (t, C-12), 37.6 (d, C-13), 75.7 (d, C-14), 38.4 (t, C-15), 82.3 (d, C-16), 64.0 (d, C-17), 69.0 (t, C-18), 55.8 (t, C-19), 42.7 (q, C-21), 56.6 (q, 1-OCH₃), 56.7 (q, 16-OCH₃).

Compound 3: N-deethyl cammaconine, which was white amorphous powder and reacted positively with bismuth potassium iodide.

¹H-NMR (CDCl₃, 400 MHz) δ: 3.38 (1H, m, H-10), 4.13 (1H, t, J=4.8 Hz, H-14β), 3.33 (1H, m, H-16a), 3.17, 3.28 (each 1H, ABq, J=11.2 Hz, H-18), 3.32, 3.35 (each 3H, s, 2×OCH₃).

¹³C-NMR (CDCl₃, 100 MHz) δ: 84.5 (d, C-1), 25.3 (t, C-2), 28.4 (t, C-3), 39.6 (s, C-4), 42.9 (d, C-5), 26.0 (t, C-6), 53.0 (d, C-7), 75.5 (s, C-8), 47.1 (d, C-9), 45.6 (d, C-10), 39.6 (s, C-11), 29.8 (t, C-12), 37.6 (d, C-13), 75.5 (d, C-14), 41.6 (t, C-15), 84.1 (d, C-16), 58.7 (d, C-17), 68.5 (t, C-18), 48.9 (t, C-19), 56.0 (q, 1-OCH₃), 56.5 (q, 16-OCH₃).

Compound 4: scaconine, which was white amorphous powder and reacted positively with bismuth potassium iodide.

¹H-NMR (CDCl₃, 400 MHz) δ: 3.04 (1H, dd, J=10.8 Hz, 6.6 Hz, H-1 μl), 3.62 (1H, t, J=4.8 Hz, H-14β), 3.15 (1H, m, H-16a), 3.17, 3.28 (each 1H, ABq, J=11.2 Hz, H-18), 0.99 (3H, t, J=7.2 Hz, NCH₂CH₃), 3.32, 3.25, 3.21 (each 3H, s, 3×OCH₃).

¹³C-NMR (CDCl₃, 400 MHz) δ: 85.7 (d, C-1), 26.3 (t, C-2), 32.2 (t, C-3), 38.8 (s, C-4), 45.6 (d, C-5), 24.9 (t, C-6), 45.4 (d, C-7), 74.2 (s, C-8), 46.3 (d, C-9), 45.7 (d, C-10), 48.7 (s, C-11), 29.4 (t, C-12), 36.8 (d, C-13), 84.4 (d, C-14), 41.6 (t, C-15), 82.6 (d, C-16), 62.4 (d, C-17), 68.7 (t, C-18), 52.9 (t, C-19), 49.3 (t, C-21), 13.5 (q, C-22), 56.2 (q, 1-OCH₃), 57.6 (q, 14-OCH₃), 56.2 (q, 16-OCH₃).

Compound 5: 8-O-methyl cammaconine, which was white amorphous powder and reacted positively with bismuth potassium iodide.

¹H-NMR (CDCl₃, 400 MHz) δ: 3.10 (1H, dd, J=10.8 Hz, 6.6 Hz, H-1 μl), 4.00 (1H, t, J=4.8 Hz, H-14β), 3.29 (1H, m, H-16a), 3.28, 3.40 (each 1H, ABq, J=11.2 Hz, H-18), 1.09 (3H, t, J=7.2 Hz, NCH₂CH₃), 3.15, 3.29, 3.37 (each 3H, s, 3×OCH₃).

¹³C-NMR (CDCl₃, 400 MHz) δ: 85.8 (d, C-1), 25.8 (t, C-2), 31.9 (t, C-3), 38.0 (s, C-4), 46.0 (d, C-5), 23.5 (t, C-6), 40.1 (d, C-7), 77.9 (s, C-8), 45.6 (d, C-9), 45.6 (d, C-10), 48.9 (s, C-11), 28.4 (t, C-12), 38.9 (d, C-13), 75.1 (d, C-14), 33.2 (t, C-15), 82.3 (d, C-16), 62.9 (d, C-17), 68.9 (t, C-18), 53.0 (t, C-19), 49.4 (t, C-21), 13.5 (q, C-22), 56.4 (q, 1-OCH₃), 48.3 (q, 8-OCH₃), 56.4 (q, 16-OCH₃).

Compound 6: 8,14-O-dimethyl cammaconine, which was white amorphous powder and reacted positively with bismuth potassium iodide.

¹H-NMR (CDCl₃, 400 MHz) δ: 3.09 (1H, dd, J=10.8 Hz, 6.6 Hz, H-1 μl), 3.55 (1H, t, J=4.8 Hz, H-14β), 3.25 (1H, m, H-16a), 3.22, 3.42 (each 1H, ABq, J=11.2 Hz, H-18), 1.07 (3H, t, J=7.2 Hz, NCH₂CH₃), 3.13, 3.29, 3.36, 3.37 (each 3H, s, 4×OCH₃).

¹³C-NMR (CDCl₃, 400 MHz) δ: 85.8 (d, C-1), 26.5 (t, C-2), 32.1 (t, C-3), 38.8 (s, C-4), 45.8 (d, C-5), 23.9 (t, C-6), 40.1 (d, C-7), 77.6 (s, C-8), 43.7 (d, C-9), 45.3 (d, C-10), 49.2 (s, C-11), 29.5 (t, C-12), 38.1 (d, C-13), 83.7 (d, C-14), 35.5 (t, C-15), 83.8 (d, C-16), 61.8 (d, C-17), 69.2 (t, C-18), 53.0 (t, C-19), 49.3 (t, C-21), 13.6 (q, C-22), 56.3 (q, 1-OCH₃), 48.1 (q, 8-OCH₃), 57.7 (q, 14-OCH₃), 56.3 (q, 16-OCH₃).

Compound 7: 16-demethyl cammaconine, which was white amorphous powder and reacted positively with bismuth potassium iodide.

¹H-NMR (CDCl₃, 400 MHz) δ: 3.10 (1H, dd, J=10.8 Hz, 6.6 Hz, H-1 μl), 4.23 (1H, t, J=4.8 Hz, H-14β), 3.82 (1H, m, H-16a), 3.19, 3.41 (each 1H, ABq, J=11.2 Hz, H-18), 1.07 (3H, t, J=7.2 Hz, NCH₂CH₃), 3.28 (3H, s, OCH₃), 4.23 (1H, t, J=4.8 Hz, H-14β).

¹³C-NMR (CDCl₃, 100 MHz) δ: 86.4 (d, C-1), 25.6 (t, C-2), 32.0 (t, C-3), 39.1 (s, C-4), 45.8 (d, C-5), 24.5 (t, C-6), 46.4 (d, C-7), 73.8 (s, C-8), 46.4 (d, C-9), 45.2 (d, C-10), 48.8 (s, C-11), 27.9 (t, C-12), 40.6 (d, C-13), 75.6 (d, C-14), 42.1 (t, C-15), 72.4 (d, C-16), 63.3 (d, C-17), 68.4 (t, C-18), 53.1 (t, C-19), 49.6 (t, C-21), 13.6 (q, C-22), 56.4 (q, 1-OCH₃).

Compound 8: 8-O-ethyl cammaconine, which was white amorphous powder and reacted positively with bismuth potassium iodide.

¹H-NMR (CDCl₃, 600 MHz) δ: 1.05 (3H, t, J=7.2 Hz, OCH₂CH₃), 1.11 (3H, t, J=7.2 Hz, NCH₂CH₃), 3.35, 3.26 (each 3H, s, 2×OCH₃).

¹³C-NMR (CDCl₃, 150 MHz) δ: 86.0 (d, C-1), 26.2 (t, C-2), 32.2 (t, C-3), 38.6 (s, C-4), 45.9 (d, C-5), 23.8 (t, C-6), 40.8 (d, C-7), 78.2 (s, C-8), 45.6 (d, C-9), 45.8 (d, C-10), 48.9 (s, C-11), 28.9 (t, C-12), 39.0 (d, C-13), 75.3 (d, C-14), 34.7 (t, C-15), 82.6 (d, C-16), 62.9 (d, C-17), 69.1 (t, C-18), 52.9 (t, C-19), 49.5 (t, C-21), 13.7 (q, C-22), 56.5 (q, 1-OCH₃), 56.5 (q, 16-OCH₃), 56.0 (t, 8-OCH₂CH₃), 16.3 (q, 8-OCH₂CH₃).

Compound 9: 8-O-ethyl, 14-O-methyl cammaconine, which was white amorphous powder and reacted positively with bismuth potassium iodide.

¹H-NMR (CDCl₃, 600 MHz) δ: 1.05 (3H, t, J=6.0 Hz, NCH₂CH₃), 3.27, 3.34, 3.36 (each 3H, s, 3×OCH₃).

¹³C-NMR (CDCl₃, 150 MHz) δ: 86.0 (d, C-1), 26.7 (t, C-2), 32.2 (t, C-3), 38.9 (s, C-4), 45.3 (d, C-5), 24.1 (t, C-6), 41.1 (d, C-7), 77.5 (s, C-8), 43.1 (d, C-9), 46.0 (d, C-10), 49.3 (s, C-11), 29.6 (t, C-12), 39.2 (d, C-13), 84.0 (d, C-14), 36.3 (t, C-15), 84.1 (d, C-16), 61.9 (d, C-17), 69.4 (t, C-18), 53.1 (t, C-19), 49.4 (t, C-21), 13.7 (q, C-22), 56.5 (q, 1-OCH₃), 57.8 (q, 14-OCH₃), 56.5 (q, 16-OCH₃), 55.5 (t, —OCH₂CH₃), 16.3 (q, OCH₂CH₃).

Compound 10: N-deethyl-N-methyl 14-O-methyl cammaconine, which was white amorphous powder and reacted positively with bismuth potassium iodide.

HR-ESI-MS m z: 408.2735 [M+H]⁺ (calcd. for C₂₃H₃₈NO₅, 408.2750); ¹H-NMR (CDCl₃, 400 MHz) δ: 3.12 (1H, m, H-1β), 4.17 (1H, t, J=4.8 Hz, H-14β), 3.38 (1H, m, H-16a), 3.25, 3.31, 3.37 (each 3H, s, 3×OCH₃), 2.30 (3H, s, NCH₃).

The beneficial effects of the present disclsoure were demonstrated in the following pharmacodynamic experiments.

### Experimental Example 1: Cardiotonic Effect of Compounds on Isolated Bullfrogs In Vitro

### 1. Experimental Method

The preparation of in vitro isolated bullfrog heart was carried out according to the method recorded in the literature (Ding Hong, Experimental Pharmacology, pp. 504-506, Science Publishing House, 2008) as follows. Bullfrogs weighing 150-200 g, male and female, were used herein. At room temperature, the brain and spinal cord of the bullfrogs was destroyed with a metal probe, and then the chest wall of the bullfrogs was incised to expose the heart and the right aorta and the left aorta were each threaded with a wire. The right aorta was knotted directly, and the left aorta was cut with scissors in a “V” shaped incision. A frog heart cannula containing a small amount of Ringer's solution was inserted into the ventricle. The wire of the left aorta was attached and secured to the lateral hook of the cannula. The blood in the heart tube of the bullfrogs was repeatedly flushed with fresh Ringer's solution until the fluid was clear and colorless. The heart cannula was inserted into the ventricle and perfused with Ringer's solution. The bullfrog heart cannula was fixed on the iron stand with a clamp. The muscle tension transducer was fixedly arranged under the bullfrog heart cannula, and the isolated bullfrog heart was connected to the muscle tension transducer via a bullfrog heart clip and a wire. The volume of solution in the cannula was adjusted by 1.0 mL and the subject compound or an equivalent volume of a solvent was added to the cannula. The subject compound was evaluated for cardiac activity on isolated perfused frog hearts, and heart rate and contraction amplitude were recorded.

The cardiotonic effects of compounds 1-10 provided in the present disclosure on isolated bullfrog hearts in vitro were investigated, with deacetylmaurolidine and mesaconitine as positive controls.

The tested concentration of compounds 1-10 was 0.005 mol/L, and the tested concentrations of the positive controls, i.e., mesaconitine and deacetylmaurolidine, were 0.01 mol/L and 0.0002 mol/L, respectively.

### 2. Experimental Results

TABLE 1
Cardiotonic effects of compounds 1-10 on isolated bullfrog hearts

| Compounds | Average increase rate (%) | Results |
|---|---|---|
| 1 | 117.6 | Strong |
| 2 | 159.3 | Strong |
| 3 | 36.8 | Significant |
| 4 | 155.9 | Strong |
| 5 | 33.6 | Significant |
| 6 | 31.2 | Significant |
| 7 | 75.2 | Strong |
| 8 | 57.2 | Significant |
| 9 | 21.8 | Moderate |
| 10 | 148.1 | Strong |
| Deacetylmaurolidine | 61 | Strong |
| Mesaconitine | 70.7 | Strong |

Noted: in table 1, the average increase rate (%) indicated the average amplitude growth rate (%) of the frog heart, and the heart-strengthening effect was categorized into the following four classes according to the magnitude: 0-15% (none); 16%-30% (+, moderate); 31%-60% (++, significant); and >60% (+++, strong).

The above experimental results showed that the compounds 1-10 of the present disclosure all had cardiotonic effects. In particular, compared with the positive control (mesaconitine), compounds 1, 2, 4, 7 and 10 of the present disclosure showed a better cardiotonic effect at lower tested concentrations, indicating that the cardiotonic effect of compounds 1, 2, 4, 7 and 10 of the present disclosure was significantly improved. The compounds of the present disclosure could be used to prepare cardiotonic drugs and drugs for preventing and/or treating heart failure.

### Experimental Example 2 Anti-Hypoxic Activity

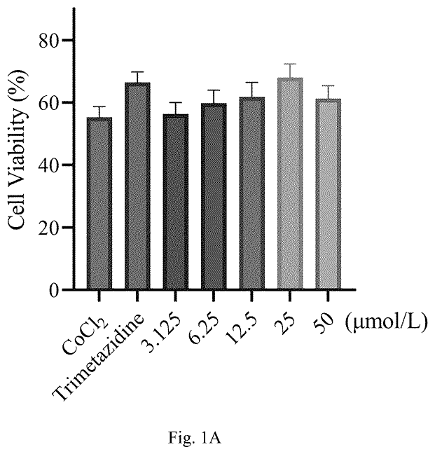

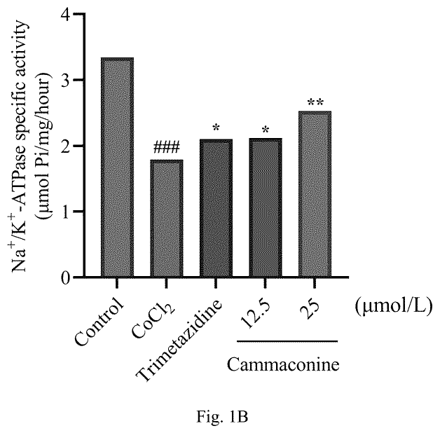

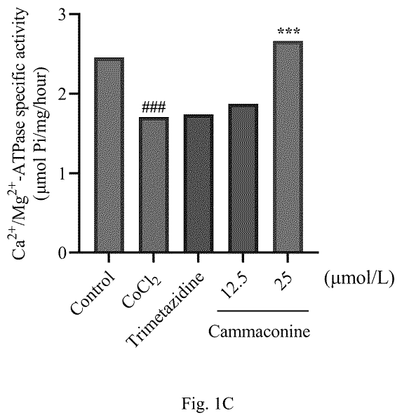

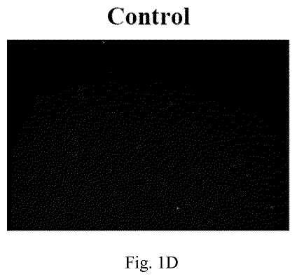

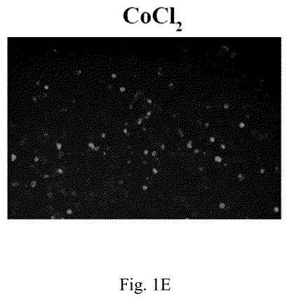

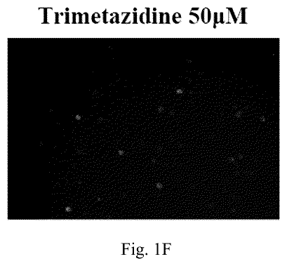

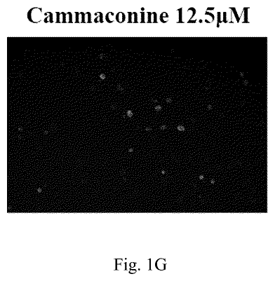

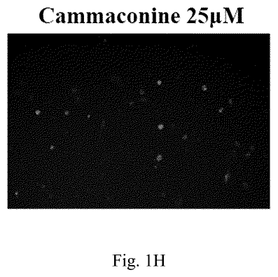

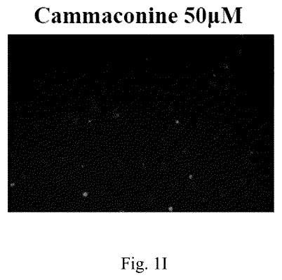

As shown in FIG. 1A, cammaconine with a concentration of 6.25-50 M had certain cardioprotective effects on H9c2 cells with myocardial hypoxia induced by CoCl₂. When cells experienced hypoxic injury, a large amount of reactive oxygen species (ROS) was released. DCFH-DA probe was used to label ROS in H9c2 cells, and 900 μM CoCl₂ and different concentrations of cammconine (12.5, 25, 50 PM) were administered to detect the changes in ROS by flow cytometry and fluorescence inverted microscopy. Results showed that cammconine significantly reduced the levels of ROS in the H9c2 cells with myocardial hypoxia induced by CoCl₂ (as shown in FIGS. 1B-1C). Changes in ATPase activity after treating the cells with myocardial hypoxia with cammaconine were detected by a kit. It was found that CoCl₂-induced hypoxia in H9c2 cells led to a activity decrease in Na⁺/K⁺-ATPase and Ca²⁺/Mg²⁺-ATPase, and cammaconine with a concentration of 25 mol/L was able to increase the enzyme activity, showing a better therapeutic effect (as shown in FIGS. 1D-I). Changes in the transcriptional level of the cardiomyocyte hypoxia key indicator HIF-1α and calcium cycle-related indicators were detected by qPCR. It was found that the mRNA transcriptional level of the HIF-1α was significantly increased after CoCl₂-induced hypoxia in H9c2 cells and decreased after cammaconine administration (as shown in FIG. 2A). CoCl₂-induced hypoxia caused changes in the transcriptional levels of mRNAs related to the calcium cycle in H9c2 cells, in which the mRNA levels of DHPR, RyR2, SERCA2a, NCX and NHE mRNAs were significantly increased, and then decreased after the drug administration (FIGS. 2B-2F). The results demonstrated that cammaconine can alleviate the hypoxic injury in cardiomyocytes to some extent.

In conclusion, the present disclosure provides a C₁₉ diterpene alkaloid of formula (I), and a preparation method and application thereof. The pharmacodynamic experiments demonstrate that the compounds of the present disclosure all have cardiotonic effects, and can be used to prepare cardiotonic drugs and drugs for preventing and/or treating heart failure. The compounds of the present disclosure can be extracted from Aconitum plants, and have broad clinical application and market prospects.

What is claimed is:

1. A compound of formula (I), or a pharmaceutically-acceptable salt thereof: 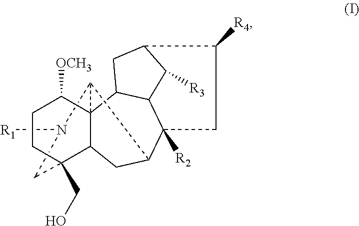
wherein R₁ is hydrogen, methyl or ethyl; R₂ is hydroxyl, methoxy or ethoxy; R₃ is hydroxyl or methoxy; and R₄ is hydroxyl or methoxy.

2. The compound of claim 1, wherein the compound is selected from the group consisting of:

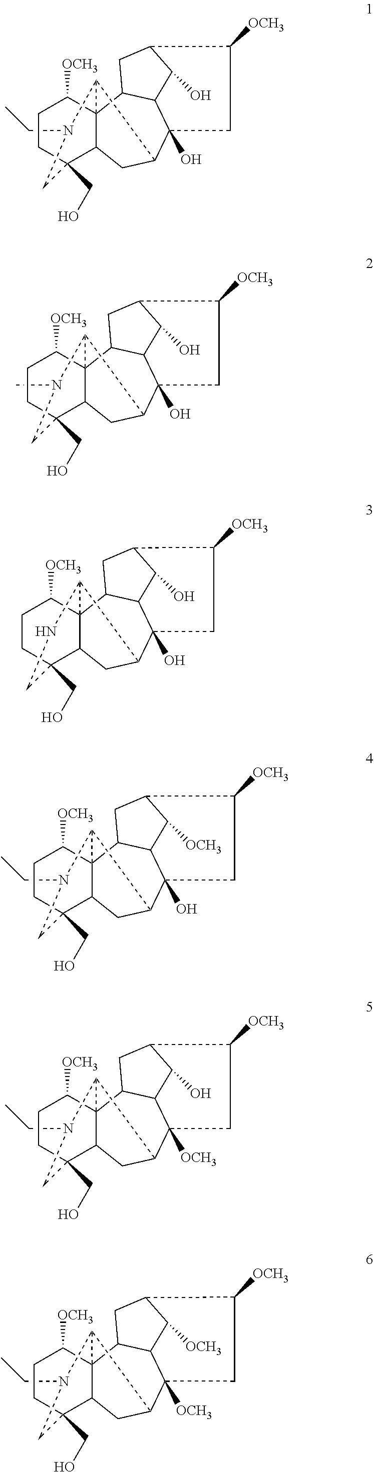

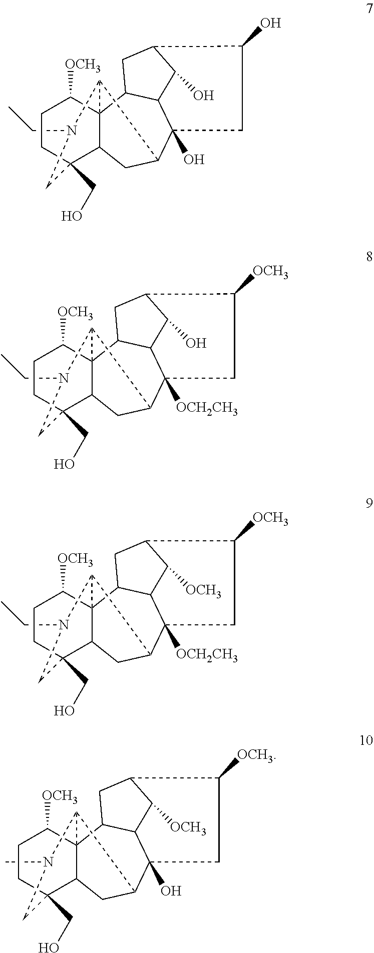

3. A method of preparing the compound of claim 1, comprising:
(a) subjecting a plant material belonging to genus Aconitum of family Ranunculaceae to extraction with ethanol, and concentration to obtain an extract;
(b) dissolving the extract in water, followed by adjustment to pH 1-3 and extraction sequentially with petroleum ether and ethyl acetate to obtain an aqueous phase;
(c) adjusting the aqueous phase to pH 9.5-11.0, followed by extraction with dichloromethane and concentration to obtain a first alkaloid sample;
(d) refluxing the first alkaloid sample with a solution of 5 wt. % NaOH in methanol for 2 h, followed by adjustment to pH 8-9 and concentration under reduced pressure to obtain a second alkaloid sample;
(e) subjecting the second alkaloids sample to normal-phase silica gel column chromatography in a gradient elution, and combining eluate fractions in sequence to respectively obtain a first crude product, a second crude product and a third crude product, wherein the eluate fractions are detected through thin-layer chromatography, and an eluent used in the normal-phase silica gel column chromatography is a mixed solution of CH₂Cl₂ and CH₃OH with a volume ratio of 1-0:0-1; and
(f) separately subjecting the first crude product, the second crude product and the third crude product to normal phase silica gel column chromatography for separation and purification to obtain the compound.

4. The method of claim 3, wherein in step (a), a concentration of the ethanol is 95 wt. %.

5. The method of claim 3, wherein in step (a), the plant material is Aconitum apetalum (Huth) B. Fedtsch.; and
in step (f), a mobile phase used in the normal phase silica gel column chromatography of the first crude product is a mixture of CH₂Cl₂, MeOH and diethylamine in a volume ratio of 100-10:1:0.1, and the obtained compound is compound 8 represented by 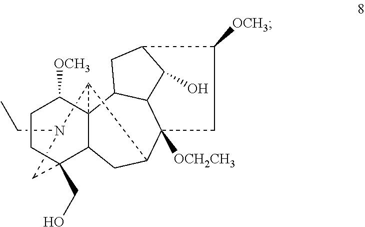
a mobile phase used in the normal phase silica gel column chromatography of the second crude product is a mixture of CH₂Cl₂, MeOH and diethylamine in a volume ratio of 100-10:1:0.1, and the obtained compound is a combination of compound 1, compound 2 and compound 3, respectively represented by 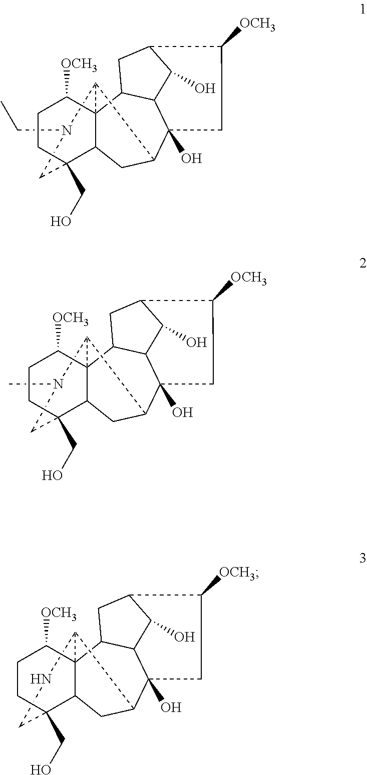
and
a mobile phase used in the normal phase silica gel column chromatography of the third crude product is a mixture of CH₂Cl₂, MeOH and diethylamine in a volume ratio of 100-10:1:0.1, and the obtained compound is compound 7 represented by

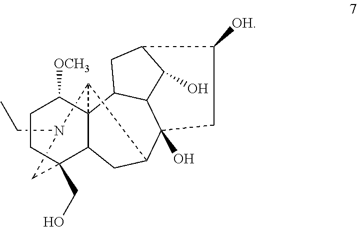

6. The method of claim 3, wherein in step (a), the plant material is Aconitum brevicalcaratum (Finet & Gagnep.) Diels;
in step (f), a mobile phase used in the normal phase silica gel column chromatography of the first crude product is a mixture of CH₂Cl₂, MeOH and diethylamine in a volume ratio of 100-10:1:0.1, and the obtained compound is a combination of compound 4, compound 5 and compound 6, respectively represented by 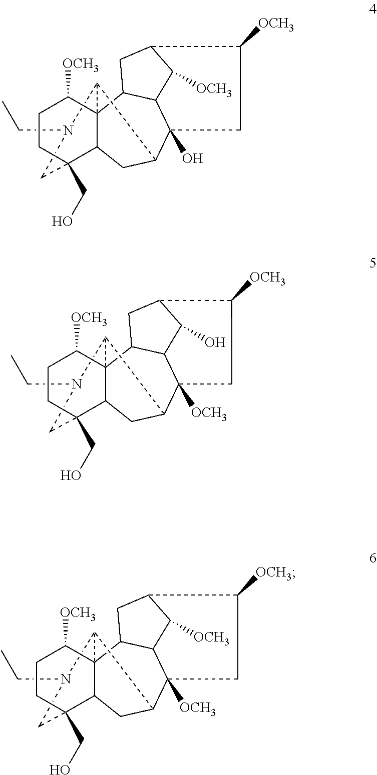
and
a mobile phase used in the normal phase silica gel column chromatography of the second crude product is a mixture of CH₂Cl₂, MeOH and diethylamine in a volume ratio of 100-10:1:0.1, and the obtained compound is a combination of compound 9 and compound 10, respectively represented by

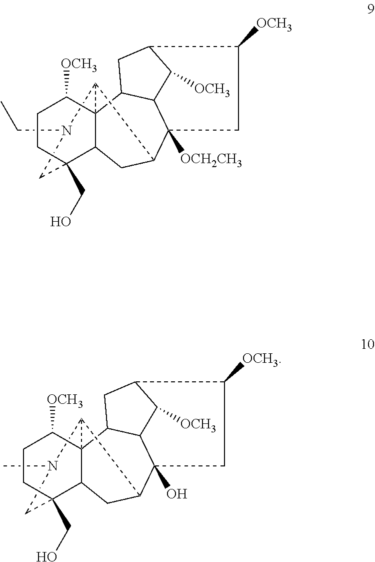

7. A pharmaceutical composition, comprising:
the compound of claim 1, or a pharmaceutically-acceptable salt thereof as an active ingredient; and
a pharmaceutically-acceptable excipient or adjuvant.

8. The pharmaceutical composition of claim 7, wherein the pharmaceutical composition is in a form of oral preparation.

9. A method for strengthening heart in a subject in need thereof, comprising:
administering a therapeutically effective amount of the compound of claim 1 or a pharmaceutically-acceptable salt thereof to the subject.

10. A method for preventing and and/or treating heart failure in a subject in need thereof, comprising:
administering a therapeutically effective amount of the compound of claim 1 or a pharmaceutically-acceptable salt thereof to the subject.
